(19) United States
(12) Patent Application Publication
(10) Pub. No.: US 2022/0096175 A1
(43) Pub. Date: Mar. 31, 2022

(54) ARTIFICIAL TRAINING DATA COLLECTION SYSTEM FOR RFID SURGICAL INSTRUMENT LOCALIZATION

(71) Applicant: Duke University, Durham, NC (US)

(72) Inventors: Ian Hill, Durham, NC (US); Patrick Codd, Durham, NC (US)

(21) Appl. No.: 17/486,369

(22) Filed: Sep. 27, 2021

Related U.S. Application Data
(60) Provisional application No. 63/083,190, filed on Sep. 25, 2020.

(51) Int. Cl. A61B 34/30; G06N 20/00; A61B 34/20

(52) U.S. Cl. CPC A61B 34/30; G06N 20/00; A61B 2034/2065; A61B 2034/2072; A61B 34/20

(57) ABSTRACT

Disclosed are systems and techniques for locating objects using machine learning algorithms. In one example, a method may include receiving at least one radio frequency signal from an electronic identification tag associated with an object. In some aspects, one or more parameters associated with the at least one RF signal can be determined. In some cases, the one or more parameters can be processed with a machine learning algorithm to determine a position of the object. In some examples, the machine learning algorithm can be trained using a position vector dataset that includes a plurality of position vectors associated with at least one signal parameter obtained using a known position of the object.

### CROSS-REFERENCE TO RELATED APPLICATIONS

This application claims the benefit of U.S. Provisional Application No. 63/083,190, filed Sep. 25, 2020, for ARTIFICIAL TRAINING DATA COLLECTION SYSTEM FOR RFID SURGICAL INSTRUMENT LOCALIZATION, which is incorporated herein by reference.

### BACKGROUND

Intraoperative surgical instrument location data is critical to many important applications in healthcare. Position data collected over a timeline describes motion, allowing for an analysis of instrument movement. Understanding instrument movement paves the way towards understanding operative approaches, motivating an optimal surgical approach with data, measuring physician prowess, automating surgical accreditation, alerting the surgical team if instruments are left inside the patient, recommending patient recovery modes from instrument dynamics, informing the design and development of new instruments, providing an operative recording of instrument positions, and mapping a surgical site.

There is currently no accurate mechanism to measure surgical instrument position in the operating room. Researchers have attempted to use video cameras, stereo vision, fluorescent labels, radio-frequency identification, and other technologies to measure the intraoperative location of surgical instruments. Each of these technologies struggle to capture accurate location data from surgical instruments due to the complexity of the operating room environment.

Surgeons, residents, and nurses huddle around the surgical site during surgery. Surgical sites are small and medical equipment surrounds the site. With bioburden, blood, and other obstructions obscuring the instruments throughout the surgery, achieving direct line of sight is difficult, especially without impeding the operation. Deterministic approaches to calculating instrument position from intraoperative sensor data have been shown to struggle in complex operating environments with high degrees of randomness. Probabilistic approaches, including Bayesian frameworks and machine learning algorithms, to predict position from variable sensor data are superior to analytical expressions relating sensor data to instrument position. However, these computational tools often require a large dataset of labeled data to train and test before they can be used to accurately locate surgical instruments intraoperatively.

Training and testing datasets are made up labeled features where the features act as predictors for the label. In the case of predicting intraoperative instrument location from sensor data, the features could be sensor signal parameters and the labels could be vector components between the sensor and the instrument. With a sufficient number of sensors, relationships between sensor signal parameters and location, and data to train and test the algorithm, predicting accurate instrument position is possible.

Collecting sufficient labeled data to train and test an algorithm in the operating room is difficult considering there is no mechanism to accurately measure intraoperative location for labeling. Therefore, it would be advantageous to collect labeled data in a way that mimics the operating environment but enables accurate position labels to use for training and testing.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter. One aspect of the present disclosure provides a method of locating objects, the method includes: receiving at least one radio frequency (RF) signal from an electronic identification tag associated with an object; determining one or more parameters associated with the at least one RF signal; and processing the one or more parameters with a machine learning algorithm to determine a position of the object.

Another aspect of the present disclosure provides an apparatus for locating objects. The apparatus comprises at least one memory, at least one transceiver, and at least one processor coupled to the at least one memory and the at least one transceiver. The at least one processor is configured to: receive, via the at least one transceiver, at least one radio frequency (RF) signal from an electronic identification tag associated with an object; determine one or more parameters associated with the at least one RF signal; and process the one or more parameters with a machine learning algorithm to determine a position of the object.

Another aspect of the present disclosure may include a non-transitory computer-readable storage medium having stored thereon instructions which, when executed by one or more processors, cause the one or more processors to: receive data associated with at least one radio frequency (RF) signal from an electronic identification tag associated with an object; determine one or more parameters associated with the at least one RF signal; and process the one or more parameters with a machine learning algorithm to determine a position of the object.

Another aspect of the present disclosure may include an apparatus for locating objects. The apparatus includes: means for receiving at least one radio frequency (RF) signal from an electronic identification tag associated with an object; means for determining one or more parameters associated with the at least one RF signal; and means for processing the one or more parameters with a machine learning algorithm to determine a position of the object.

Another aspect of the present disclosure provides a method for training a machine learning algorithm, the method includes: positioning an object having at least one electronic identification tag at a plurality of positions relative to at least one electronic identification tag reader; determining, based on data obtained using the at least one electronic identification tag reader, one or more signal parameters corresponding to each of the plurality of positions; and associating each of the one or more signal parameters with one or more position vectors to yield a position vector dataset, wherein each of the one or more position vectors corresponds to a respective position from the plurality of positions relative to a position associated with the at least one electronic identification tag reader.

Another aspect of the present disclosure provides an apparatus for training a machine learning algorithm. The apparatus comprises at least one memory and at least one processor coupled to the at least one memory. The at least one processor is configured to: position an object having at least one electronic identification tag at a plurality of positions relative to at least one electronic identification tag reader; determine one or more signal parameters corresponding to each of the plurality of positions; and associate each of the one or more signal parameters with one or more position vectors to yield a position vector dataset, wherein each of the one or more position vectors corresponds to a respective position from the plurality of positions relative to a position associated with the at least one electronic identification tag reader.

Another aspect of the present disclosure may include a non-transitory computer-readable storage medium having stored thereon instructions which, when executed by one or more processors, cause the one or more processors to: position an object having at least one electronic identification tag at a plurality of positions relative to at least one electronic identification tag reader; determine one or more signal parameters corresponding to each of the plurality of positions; and associate each of the one or more signal parameters with one or more position vectors to yield a position vector dataset, wherein each of the one or more position vectors corresponds to a respective position from the plurality of positions relative to a position associated with the at least one electronic identification tag reader.

Another aspect of the present disclosure may include an apparatus for training a machine learning algorithm. The apparatus includes: means for positioning an object having at least one electronic identification tag at a plurality of positions relative to at least one electronic identification tag reader; means for determining, based on data obtained using the at least one electronic identification tag reader, one or more signal parameters corresponding to each of the plurality of positions; and means for associating each of the one or more signal parameters with one or more position vectors to yield a position vector dataset, wherein each of the one or more position vectors corresponds to a respective position from the plurality of positions relative to a position associated with the at least one electronic identification tag reader.

Another aspect of the present disclosure provides a method for locating objects, the method includes: moving an object to a position using at least one positioner; obtaining sensor data from the object at the position using at least one sensor; and associating the sensor data from the object with location data corresponding to the position to yield location-labeled sensor data.

Another aspect of the present disclosure provides an apparatus for locating objects. The apparatus comprises at least one memory, at least one sensor, at least one positioner, and at least one processor coupled to the at least one memory, the at least one sensor, and the at least one positioner. The at least one processor is configured to: move an object to a position using the at least one positioner; obtain sensor data from the object at the position using the at least one sensor; and associate the senor data from the object with location data corresponding to the position to yield location-labeled sensor data.

Another aspect of the present disclosure may include a non-transitory computer-readable storage medium having stored thereon instructions which, when executed by one or more processors, cause the one or more processors to: move an object to a position; obtain sensor data from the object at the position; and associate the senor data from the object with location data corresponding to the position to yield location-labeled sensor data.

Another aspect of the present disclosure may include an apparatus for locating objects. The apparatus includes: means for moving an object to a position; means for obtaining sensor data from the object at the position; and means for associating the sensor data from the object with location data corresponding to the position to yield location-labeled sensor data

These and other aspects will be described more fully with reference to the Figures and Examples disclosed herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1 is a top diagram view of an example environment in which a system in accordance with aspects of the present disclosure may be implemented.

FIG. 2 is a system diagram illustrating aspects of the present disclosure.

FIG. 3 is another system diagram illustrating aspects of the present disclosure.

FIG. 4 is a flowchart illustrating an example method for locating objects.

FIG. 5 is a flowchart illustrating another example method for locating objects.

FIG. 6 is a flowchart illustrating an example method for training a machine learning algorithm.

FIG. 7 is a flowchart illustrating another example method for training a locating objects.

FIG. 8 illustrates an example computing device in accordance with some examples.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

Unless otherwise defined, all technical terms used herein have the same meaning as commonly understood by one of ordinary skill in the art to which this disclosure belongs.

Localization of surgical instruments via RFID has been historically challenging, based on the difficulty of deterministically computing a location based on signal parameters (frequency, phase, and/or return signal strength) due to factors such as high signal to noise ratios, multipath error, and/or line of sight (LOS)/NLOS variation. In some cases, computational models that identify patterns in input features in order to localize instruments may be used. However, clinical localization data remains difficult to achieve.

The localization problem can be defined by the computation of the vector from each reader antenna to each instrument, where only a few instruments are in the field at once. This is a relative localization problem, as the absolute position of the reader antennas is unknown. The absolute location is of little consequence as the ultimate reference position for a surgery is the center of the surgical site, which is unique to each operation. Transient change in instrument position is the ultimate value proposition of relative localization as it can be used to understand surgeon movement, gauge surgical efficacy, and predict outcomes.

The present disclosure provides systems and techniques for locating medical instruments using a machine learning algorithm and for training the machine learning algorithm. In some aspects, the present disclosure provides a data collection system that automatically labels RFID-read data with corresponding localization vectors. Those of skill in the art will recognize that RFID may be construed broadly to encompass a variety of technologies that allow a device, commonly referred to as a tag, to be wirelessly read, identified, and/or located in space. In some cases, the systems and techniques described herein can be used for expedient generation of a large body of artificial data that can be used to pre-train machine learning models that predict localization vectors from RFID-read data.

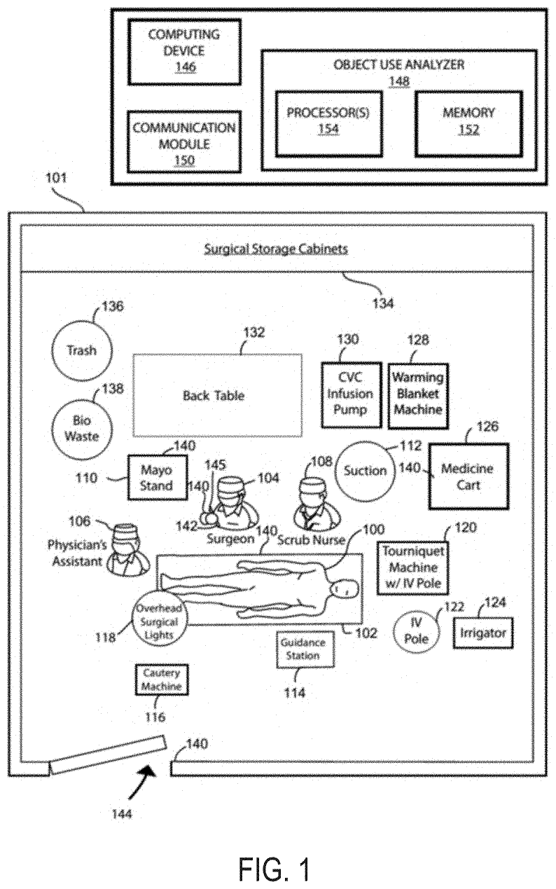

FIG. 1 illustrates a top diagram view of an example environment (e.g., Operating Room (OR) 101) in which a system in accordance with embodiments of the present disclosure may be implemented. It is noted that the system is described in this example as being implemented in an OR, although the system may alternatively be implemented in any other suitable environment such as a factory, dentist office, veterinary clinic, or kitchen. Further, it is noted that in this example, the placement of a patient, medical practitioners, and medical equipment are shown during surgery.

Referring to FIG. 1, a patient 100 is positioned on a surgical table 102. Further, medical practitioners, including a surgeon 104, an assistant 106, and a scrub nurse 108, are shown positioned about the patient 100 for performing the surgery. Other medical practitioners may also be present in the OR 101, but only these 3 medical practitioners are shown in this example for convenience of illustration.

Various medical equipment and other objects may be located in the OR 101 during the surgery. For example, a Mayo stand 110, a suction machine 112, a guidance station 114, a cautery machine 116, surgical lights 118, a tourniquet machine 120, an intravenous (IV) pole 122, an irrigator 124, a medicine cart 126, a warming blanket machine 128, a CVC infusion pump 130, and/or various other medical equipment may be located in the OR 101. The OR 101 may also include a back table 132, various cabinets 134, and other equipment for carrying or storing medical equipment and supplies. Further, the OR 101 may include various disposal containers such a trash bin 136 and a biologics waste bin 138.

In accordance with some embodiments, various RFID readers and tags may be distributed within the OR 101. For convenience of illustration, the location of placement of RFID readers and RFID tags are indicated by reference numbers 140 and 142, respectively. In this example, RFID readers 140 are attached to the Mayo stand, the surgical table 102, a sleeve of the surgeon 104, and a doorway 144 to the OR 101. It should be understood that the location of these RFID readers 140 are only examples and should not be considered limiting as the RFID readers may be attached to other medical equipment or objects in the OR 101 or another environment. It should also be noted that one or more RFID readers may be attached to a particular object or location. For example, multiple RFID readers may be attached to the Mayo stand 110 and the surgical table 102.

An RFID tag 142 may be attached to medical equipment or other objects for tracking and management of the medical equipment and/or objects in accordance with embodiments of the present disclosure. In this example, an RFID tag 142 is attached to the non-working end of a surgical instrument 145. RFID readers 140 in the OR 101 may detect that the surgical instrument 145 is nearby to thereby track usage of the surgical instrument 145. For example, the surgical instrument 145 may be placed in a tray on the Mayo stand 110 during preparation for the surgery on the patient 100. The RFID reader 140 on the Mayo stand 110 may interrogate the RFID tag 142 attached to the surgical instrument 145 to acquire an ID of the surgical instrument 145. The ID may be acquired when the surgical instrument 145 is sufficiently close to the Mayo stand's 110 RFID reader 140. In this way, it may be determined that the surgical instrument 145 was provided for the surgery. Also, the Mayo stand's 110 RFID reader 140 may fail to interrogate the RFID reader 140 in cases in which the surgical instrument's 145 RFID tag 142 is out of range. The detection of a RFID tag 142 within communicated range is information indicative of the presence of the associated medical equipment within a predetermined area, such as on the Mayo stand 110.

It is noted that an RFID reader's field of view is dependent upon the pairing of its antennas. The range of the RFID reader is based upon its antennas and the antennas can have different fields of view. The combination of these fields of view determines where it can read RFID tags.

It is noted that this example and others throughout refer to use of RFID readers and RFID tags. However, this should not be considered limiting. When suitable, any other type of electronic identification readers and tags may be utilized.

The Mayo stand's 110 RFID reader 140 and other readers in the OR 101 may communicate acquired IDs of nearby medical equipment to a computing device 146 for analysis of the usage of medical equipment. For example, the computing device 146 may include an object use analyzer 148 configured to receive, from the RFID readers 140, information indicating presence of RFID tags 142 within areas near the respective RFID readers 140. These areas may be referred to as “predetermined areas,” because placement of the RFID readers 140 within the OR 101 is known or recognized by the object use analyzer 148. Thereby, when a RFID reader 140 detects presence of a RFID tag 142, the ID of the RFID tag 142 (which identifies the medical equipment the RFID tag 142 is attached to) is communicated to a communication module 150 of the computing device 146. In this way, the object use analyzer 148 can be informed that the medical equipment associated with the ID was at the predetermined area of the RFID reader 140 or at a distance away from the predetermined area inferred from the power of the receive signal. For example, the object use analyzer 148 can know or recognize that the surgical instrument 145 is within a predetermined area of the RFID reader 140 of the Mayo stand 110. Conversely, if the RFID tag 142 of the surgical instrument 145 is not detected by the RFID reader 140 of the Mayo stand 110, the object use analyzer 148 can know or recognize that the surgical instrument 145 is not within the predetermined area of the RFID reader 140 of the Mayo stand 110.

The RFID reader, such as the RFID readers 140 shown in FIG. 1, may stream tag read data over an IP port that can be read by a remote listening computer. The port number and TCP port number are predetermined to provide a wireless communication link between the two without physical tethering. The receiving computer may be located in the OR 101 or outside the OR 101. Data can also be sent and received over Ethernet or USB.

Data about the presence of RFID tags 142 at predetermined areas of the RFID readers 140 can be used to analyze usage of medical equipment. For example, multiple different types of surgical instruments may have RFID tags 142 attached to them. These RFID tags 142 may each have IDs that uniquely identify the surgical instrument it is attached to. The object use analyzer 148 may include a database that can be used to associate an ID with a particular type of surgical instrument. Prior to beginning a surgery, the surgical instruments may be brought into the OR 101 on a tray placed onto the Mayo stand 110. An RFID reader on the tray and/or the RFID reader 140 on the Mayo stand 110 may read each RFID tag attached to the surgical instruments. The ID of each read RFID tag may be communicated to the object use analyzer 148 for determining their presence and availability for use during the surgery. In this way, each surgical instrument made available for the surgery by the surgeon 104 can be tracked and recorded in a suitable database.

Continuing the aforementioned example, the surgeon 104 may begin the surgery and begin utilizing a surgical instrument, such as a scalpel. The RFID reader 140 at the stand may continuously poll RFID tags and reported identified RFID tags to the object use analyzer 148 of the computing device 146. The object use analyzer 148 may recognize that the RFID tag of the surgical instrument is not identified, and therefore assume that it has been removed from the surgical tray and being used for the surgery. The object use analyzer 148 may also track whether the surgical instrument is returned to the surgical tray. In this way, the object use analyzer 148 may track usage of surgical instruments based on whether they are detected by the RFID reader 140 attached to the Mayo stand 110.

It is noted that the object use analyzer 148 may include any suitable hardware, software, firmware, or combinations thereof for implementing the functionality described herein. For example, the object use analyzer 148 may include memory 152 and one or more processors 154 for implementing the functionality described herein. It is also noted that the functionality described herein may be implemented by the object use analyzer 148 alone, together with one or more other computing devices, or separately by an object use analyzer of one or more other computing devices.

Further, it is noted that although electronic identification tags and readers (e.g., RFID tags and readers) are described as being used to track medical equipment, it should be understood that other suitable systems and techniques may be used for tracking medical equipment, such as the presence of medical equipment within a predetermined area. For example, other tracking modalities that may be used together with the electronic identification tags and readers to acquire tracking information include, but are not limited to, visible light cameras, magnetic field detectors, and the like. Tracking information acquired by such technology may be communicated to object use analyzers as disclosed herein for use in analyzing medical equipment usage and other disclosed methods.

Referring to FIG. 1, aside from placement at the Mayo stand 110, RFID readers 140 are also shown in the figure as being placed in other locations throughout the OR 101. For example, RFID readers 140 are shown as being placed at on the operating table 102, on the surgeon's 104 sleeve, and the doorway 144. In one illustrative example, the surgeon 104 can wear an electronic identification tag (e.g., RFID reader 140) that can be used to enable intraoperative localization of the wrist, which could be used to determine individual that is performing certain tasks (e.g., operating, using instruments, etc.).

Further, it is noted that the RFID readers may also be placed at other locations throughout the OR 101 for reading RFID tags attached to medical equipment to thereby track and locate the medical equipment. Placement of RFID readers 140 throughout the OR 101 can be used for determining the presence of medical equipment in these areas to thereby deduce a use of the medical equipment, such as the described example of the use of the surgical instrument 145 if it is determined that it is no longer present at the Mayo stand 110. For example, placing an RFID reader and antenna with field of view tuned to view the doorway of the operating room can be used to know exactly what instruments enter the room. Knowing the objects that entered the room can be used for cost recording, as CPT codes can be automatically called.

Some antenna characteristics of RFID readers that can be important to the uses disclosed herein include frequency, gain, polarization, and form factor. For applications disclosed herein, an ultra-high frequency, high gain, circularly polarized, mat antenna may be used. There are three classes of RFID frequencies: low frequency (LF), high frequency (HF), and UHF. UHF can provide the longest read range among these three and may be utilized for the applications and examples disclosed herein. Understanding that small sized RFID tags may need to be used to fit some medical equipment such as surgical instruments, UHF may be used to provide the longest read range of the three. A mixture of high and low gain reader antennas may be utilized as they allow for either longer communication range and limited span of the signal or vice versa.

In some aspects, two classes of polarized antennas may be used: circular and linear. Linear polarization can allow for longer read ranges, but tags need to be aligned to the signal propagation. Circularly-polarized antennas may be used in examples disclosed herein as surgical tool orientation is random in an OR.

In some examples, the form factor of antennas may be a mat that can be laid underneath a sterile field, patient, instrument tables, central sterilization and processing tables, and require little space. Their positioning and power tuning allow for a limited field of view encompassing only instruments that enter their radiation field. This characteristic may be desirable because instruments can be read by an antenna focused on the surgical site, whereas instruments that are on back tables cannot be read. For tool counting within trays or across the larger area of a table away from the surgical site, an unfocused antenna may be desirable. This type of setup allows for detection of the device within the field of interest.

When an instrument is detected within a field of interest via an RFID tag read, it may be referred to as an “instrument read”. Instrument reads that are obtained by the antenna focused on the surgical site (e.g., surgical table 102) may be marked as “used instruments” and others being read on instrument tables are not. Some usage statistics may also be inferred from the lack of instrument reads in a particular field.

In accordance with embodiments, mat antennas may be placed under surgical drapes, on a Mayo stand, on instrument back tables, or anywhere else relevant within the OR 101 or within the workflow of sterilization and transportation of medical equipment (e.g., surgical instruments) for real-time or near real-time medical instrument census and counts in those areas. Placement in doorways (e.g., doorway 144) can provide information on the medical equipment contained in a room. Central sterilization and processing (CSP) may implement antennas for censusing trays at the point of entry and exit to ensure their contents are correct or as expected. The UHF RFID reader may contain multiple antenna ports for communication with multiple antennae at unique or overlapping areas of interest (e.g., the surgical site, Mayo stand, and back tables). The reader may connect to software or other enabling technology that controls power to each antenna and other pertinent RFID settings (such as Gen2 air interface protocol settings), tunable for precise read rate and range. Suitable communication systems, such as a computer, may subsequently broadcast usage data of an Internet protocol (IP) port to be read by a computing device, such as computing device 146. The data may be saved locally, saved to a cloud-based database, or otherwise suitably logged. The data may be manipulated as needed to derive statistics prior to logging or being stored.

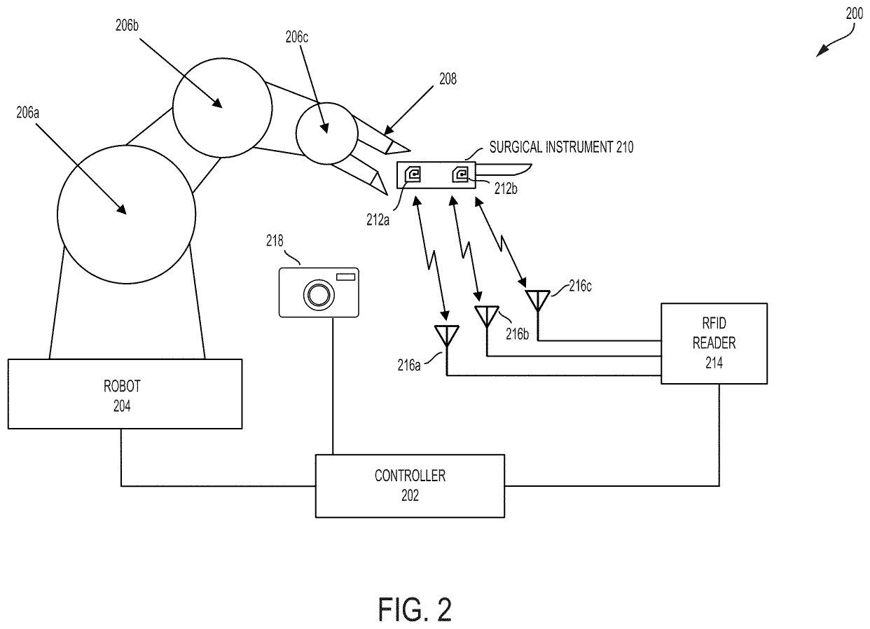

FIG. 2 illustrates a system 200 for training a machine learning algorithm to detect and locate objects using radio frequency identification (RFID), in accordance with some aspects of the present disclosure. In some cases, system 200 can be designed to mimic a surgical environment such as OR 101. In some examples, system 200 can include a controller 202 that includes one or more processors that can be configured to implement a machine learning algorithm. In some cases, the machine learning algorithm can include a Gaussian Process Regression algorithm in which predictions that are made by the algorithm can inherently provide confidence intervals.

In some examples, controller 202 can be communicatively coupled to robot 204. In some cases, robot 204 may include a robotic arm having one or more joints (e.g., joints 206a, 206b, and 206c). In some embodiments, robot 204 may include a gripping mechanism at the end of the robotic arm such as end effector 208. In some cases, end effector 208 can be configured to hold an object such as surgical instrument 210. Although surgical instrument 210 is illustrated as a scalpel, surgical instrument 210 may include any other object or medical device.

In some aspects, robot 204 can correspond to a 3D positioning robot that can be used to move surgical instrument 210 to one or more locations within a 3-dimensional space. In some cases, the orientation and position of end effector 208 is controlled (e.g., by controller 202) to move surgical instrument 210 to random positions and/or predetermined positions in a semi-spherical space.

In some examples, system 200 can include an RFID reader 214 that may include or be coupled to one or more antennas 216a, 216b, and 216c. In some cases, antennas 216a, 216b, and 216c can include linear-polarized antennas, circular-polarized antennas, slant-polarized antennas, phased antenna arrays, any other type of antennas, and/or any combination thereof. In some embodiments, the antennas 216a, 216b, and 216c may be configured to be a specific distance and/or orientation from each other (e.g., in multiple planes or co-planar). Although system 300 is illustrated as having 3 antennas, the present technology may be implemented using any number of antennas.

In some embodiments, surgical instrument 210 can include one or more electronic identification tags (e.g., RFID tag 212a and RFID tag 212b). For instance, RFID tag 212a and/or RFID tag 212b may be attached, connected, and/or embedded with surgical instrument 210. In some examples, RFID reader 214 may transmit and receive one or more RF signals (e.g., via antennas 216a, 216b, and 216c) that can be used to read, track, identify, trigger, and/or otherwise communicate with RFID tag 212a and/or RFID tag 212b on surgical instrument 210.

In some aspects, RFID reader 214 can obtain one or more parameters (e.g., RFID read data) from RFID tag 212a and/or RFID tag 212b. For example, the one or more parameters can include an electronic product code (EPC), an instrument geometry identifier, a received signal strength indicator (RSSI), a phase, a frequency, and/or an antenna number. In some cases, each of these parameters can be used to describe patterns in the read data that can affect localization of surgical instrument 210.

In some embodiments, the EPC can be used to train a machine learning model with individual instrument readability biases (e.g., RFID tag 212a and/or RFID tag 212b may have different readability that may impact signal parameters). In some cases, unique instrument profiles may cause an RFID tag (e.g., RFID tag 212a) to protrude more than others, which may offer enhanced readability. In some instances, different RFID tags may inherently have different sensitivity. Furthermore, the size, shape, and position of RFID tag 212a and/or RFID tag 212b on surgical instrument 210 may affect how well the tag responds to RF signals. In some aspects, the geometry identifier may be used to address instrument group biases. For example, instruments may be grouped into different bins that may be associated with different aspect ratios.

In some aspects, the RSSI parameter (e.g., associated with RFID tag 212a and/or RFID tag 212b) can be used to determine power ranging inference. In some cases, the phase parameter can be used to determine orientation and/or Mod 2π ranging. In some examples, the frequency parameter can be used to determine time of flight (ToF) and/or time difference of arrival (TDOA) between antennas.

In some embodiments, each of the parameters obtained from RFID tag 212a and/or RFID tag 212b can be associated with a position vector that relates the position of an RFID tag to a respective antenna. For example, antenna 216a can be used to obtain an RSSI value from RFID tag 212a and the RSSI value can be associated with a position vector relating the position of antenna 216a to the position of RFID tag 212a.

In some examples, the position of an RFID tag (e.g., RFID tag 212a) can be determined based on the position of robot 204. For instance, the robotic arm length and motor positions can be used to calculate the position vectors between RFID tags and the antennas (e.g., antennas are stationary). In one illustrative example, electronically-controlled motors (e.g., Arduino-controlled stepper motors) in the arm of robot 204 and linkage lengths (e.g., 60 cm total length) can be used to calculate position vectors between the instrument-tag pair (e.g., RFID tag 212a and/or 212b on surgical instrument 210) and each antenna (e.g., antenna 216a, 216b, and/or 216c). In some configurations, a clock signal associated with RFID reader 214 may be synchronized with a clock signal associated with the robot controller (e.g., controller 202) such that RFID read data can be automatically labeled with position vectors.

In some cases, system 200 can include one or more other sensors that can be used to collect data associated with surgical instrument 210 at one or more different positions. For example, system 200 may include a camera 218 that may be communicatively coupled to controller 202. In some aspects, camera 218 may capture image data and/or video data associated with surgical instruments 210. In some examples, data captured by camera 218 may be associated with a position vector that relates the position of an RFID tag to a respective antenna. In some aspects, data captured by camera 218 may also be associated with one or more RFID parameters captured at the same position (e.g., associated with a same position vector). In some cases, data captured by camera 218 may be used to train a machine learning algorithm to detect and/or locate surgical instrument 210. In some examples, positions of robot 204 can be calibrated using data from camera 218 and/or from any other sensors (e.g., stereo vision, infrared camera, etc.).

Although robot 204 is illustrated as a linkage-type robot having a robotic arm and multiple joints, alternative implementations for positioning surgical instrument 210 may be used in accordance with the present technology. For example, in some aspects, robot 204 can correspond to a string localizer that includes one or more stepper motors and spools of string that may be tied to an object to adjust the object's position and/or orientation. In some cases, a string localizer may be used to implement the present technology to reduce metal in the environment (e.g., reduce interference to RF signals).

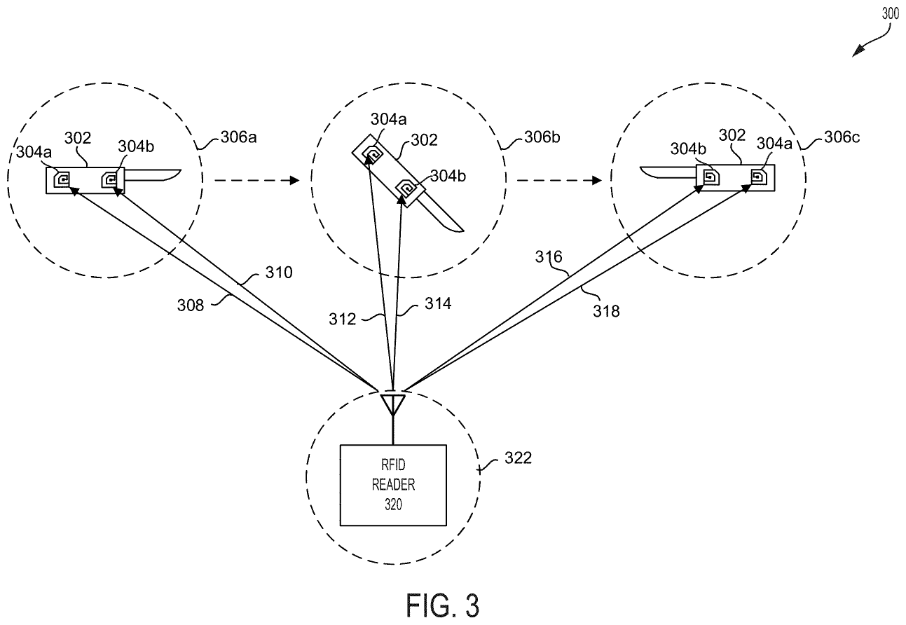

FIG. 3 illustrates a system 300 for training a machine learning algorithm to detect and locate objects using radio frequency identification (RFID), in accordance with some aspects of the present disclosure. System 300 may include one or more RFID readers such as RFID reader 320. In some aspects, RFID reader 320 may be located at position 322. In some configurations, the position 322 of RFID reader 320 may be fixed or stationary.

In some embodiments, RFID reader 320 can transmit and receive radio frequency signals that can be used to communicate with one or more RFID tags that are associated with one or more objects. For example, RFID reader 320 can be used to obtain RFID data from RFID tag 304a and/or RFID tag 304b. In some cases, RFID tag 304a and/or RFID tag 304b may be associated (e.g., attached, connected, embedded, etc.) with surgical instrument 302.

In some aspects, surgical instrument 302 can be moved to different positions that are within range of RFID reader 320. For example, a robot (e.g., robot 204) can be used to move surgical instrument 302 to one or more random positions and/or preconfigured positions. In some cases, the orientation of surgical instrument 302 may also be changed (e.g., at the same position or at different positions). For example, surgical instrument 320 can be rotated around an axis at a stationary position. As illustrated in FIG. 3, surgical instrument 302 is first located at position 306a with the blade at approximately a 0-degree orientation. In the second iteration, surgical instrument 302 is located at position 306b with the blade at approximately a 315-degree orientation. In the third iteration, surgical instrument 302 is located at position 306c with the blade at approximately a 180-degree orientation (e.g., mirrored from orientation in position 306a).

In some examples, RFID reader 320 can read or obtain one or more parameters associated with RFID tag 304a and/or RFID tag 304b when surgical instrument 302 is located at each of positions 306a, 306b, and 306c. In some cases, the one or more parameters can include an electronic product code (EPC), an instrument geometry identifier, a received signal strength indicator (RSSI), a phase, a frequency, and/or an antenna number.

In some embodiments, each of the parameters obtained from RFID tag 304a and/or RFID tag 304b can be associated with a position vector that relates the position of an RFID tag to the position 322 of RFID reader 320. For example, position vector 308 can relate the position 322 of RFID reader 320 with the position 306a of RFID tag 304a. Similarly, position vector 310 can relate the position 322 of RFID reader 320 with the position 306a of RFID tag 304b. In some examples, the parameters obtained from RFID tag 304a and RFID tag 304b while located at position 306a can be associated with position vector 308 and position vector 310, respectively.

In another example, position vector 312 can relate the position 322 of RFID reader 320 with the position 306b of RFID tag 304a. Similarly, position vector 314 can relate the position 322 of RFID reader 320 with the position 306b of RFID tag 304b. In some examples, the parameters obtained from RFID tag 304a and RFID tag 304b while located at position 306b can be associated with position vector 312 and position vector 314, respectively.

In another example, position vector 316 can relate the position 322 of RFID reader 320 with the position 306c of RFID tag 304b. Similarly, position vector 318 can relate the position 322 of RFID reader 320 with the position 306c of RFID tag 304a. In some examples, the parameters obtained from RFID tag 304a and RFID tag 304b while located at position 306c can be associated with position vector 318 and position vector 316, respectively.

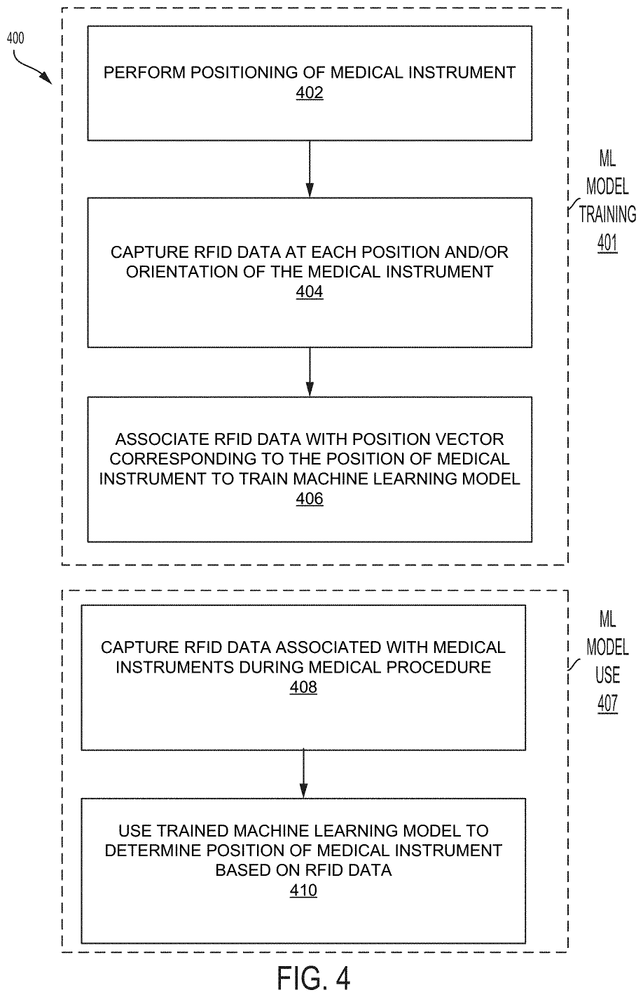

FIG. 4 illustrates an example method 400 for training and implementing a machine learning algorithm to locate objects. In some aspects, method 400 can include process 401 that can correspond to machine learning (ML) model training. In some examples, method 400 can include process 407 that can correspond to implementation (e.g., use) of the trained machine learning model. At block 402, the ML training process 401 can include performing positioning (e.g., random positioning and/or preconfigured positioning) of a medical instrument. In some examples, the random positioning can be performed using a robotic arm (e.g., robot 204). At block 404, the ML training process 401 can include capturing RFID data at each position and/or orientation of the medical instrument. For example, RFID reader 320 can capture RFID data associated with surgical instrument 302 at positions 306a, 306b, and 306c.

At block 406, the ML training process 401 can include associating RFID data with a position vector corresponding to the position of the medical instrument in order to train the machine learning model. In some cases, the position vector can correspond to the position of the medical instrument relative to the RFID reader. In some cases, the position of the medical instrument can be determined based on the settings, configuration, and/or specifications of the positioning robot. In some examples, the position of the RFID reader can be fixed. For instance, RFID reader 320 can be fixed at position 322 and position vector 308 can correspond to the position of RFID tag 304a at position 306a relative to RFID reader 320. In some examples, ML training process 401 may be repeated until the machine learning algorithm is trained (e.g., algorithm can determine position of instrument based on RFID data).

In some embodiments, once a machine learning model is trained to predict object location from RFID parameters, the model can be applied to RFID data collected from real medical procedures (e.g., surgeries). The machine learning model can provide a framework for localizing surgical instruments autonomously without impacting surgical workflow. For example, at block 408 the ML model can be used to capture RFID data associated with medical instruments during a medical procedure. In some cases, the ML system may be calibrated prior to commencing a medical procedure (e.g., by placing a well-characterized tagged instrument at predetermined locations before surgery). In some examples, the RFID data can be captured using RFID readers 140 in OR 101. In some cases, the RFID data can include an electronic product code (EPC), an instrument geometry identifier, a received signal strength indicator (RSSI), a phase, a frequency, and/or an antenna number. In some cases, the

At block 410, the process 400 can include using the trained machine learning model to determine the position of medical instruments based on RFID data. For instance, the trained machine learning algorithm can use RFID data to determine position vectors that provide the location of the medical instrument(s) relative to one or more RFID readers. In some examples, the ML algorithm can provide a confidence interval that is associated with the determined location. In some cases, knowing the location of surgical tools can help speed up surgeries by reducing the time spent looking for specific tools, which can also save time and operating room costs. In some examples, a log or history of instrument positions over time can be used to calculate time derivatives of location (e.g., velocity, acceleration, jerk, etc.). In some embodiments, the location of the instrument over time can be used to eliminate predicted location candidates by stipulating linear motion.

In some examples, the medical instrument can be identified based on time derivatives of predicted location (e.g., how the instrument moves). In some cases, the type of surgery may be determined based on the type of instruments used, instrument use durations, instrument locations, and/or time derivatives of instrument locations. In some configurations, the duration of a surgical procedure can be predicted based on instrument locations, durations of use, and time derivatives of locations.

In some examples, one or more medical professionals (e.g., surgeon, resident, nurse, etc.) may also wear or otherwise be associated with RFID tags. In some cases, these tags may be located near the hands of the medical professional and can be localized using the present technology. In some aspects, the RFID system can be used to record actions by different individuals (e.g., determine which doctor is operating with what instrument by comparing the location of the instrument and the location of the hand). In some cases, the locations of the surgeons' hands can be used to evaluate who was operating at what time and/or for what portion of the surgery. In some examples, the time derivatives of location can be used to evaluate surgical prowess (e.g., calculate a metric for individual surgeons based on instrument use and movement that can be used to evaluate skill). In some cases, surgical technique based on time derivatives of location can be used to train new surgeons and/or inform an optimal approach for a procedure. In some examples, transient locations and their time derivatives can be used to train robots to perform medical procedures. In some embodiments, the portion of resident operating time and instrument kinematics can be used to inform skill level and/or preparedness.

In some aspects, the optimal medication and recovery of a patient can be determined based on type of instruments used and duration of use. In some examples, instrument kinematics can be used to inform design of new instruments. In some embodiments, instrument locations, durations of use, and kinematics can be used to demonstrate level care (e.g., determine whether standard procedures/protocol were followed). In some cases, instrument locations can be used to predict forthcoming need for supplies. In some examples, instrument locations can be used to map a surgical site.

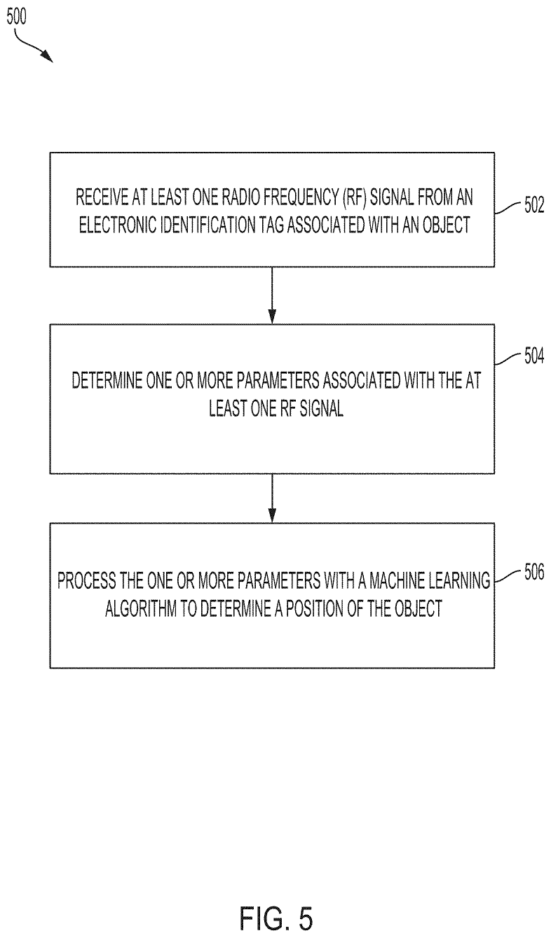

FIG. 5 illustrates an example method 500 for locating objects using a machine learning algorithm. At block 502, the method 500 includes receiving at least one radio frequency (RF) signal from an electronic identification tag associated with an object. In some aspects, the electronic identification tag may include a radio frequency identification (RFID) tag. For example, RFID reader 140 can receive at least one RF signal from RFID tag 142 that is associated with surgical instrument 145. At block 504, the method 500 includes determining one or more parameters associated with the at least one RF signal. In some aspects, the one or more parameters can include at least one of a phase, a frequency, a received signal strength indicator (RSSI), a time of flight (ToF), an Electronic Product Code (EPC), and an instrument geometry identifier. For example, object use analyzer 148 can determine one or more parameters that are associated with an RF signal received from RFID tag 142.

At block 506, the method 500 includes processing the one or more parameters with a machine learning algorithm to determine a position of the object. In some aspects, the object can include at least one of a medical device and a surgical instrument, wherein the object is within an operating room environment. For example, object use analyzer 148 may implement a machine learning algorithm to determine a position of surgical instrument 145 within OR 101. In some examples, the machine learning algorithm can correspond to a Gaussian Process Regression algorithm.

In some embodiments, the machine learning algorithm can be trained using a position vector dataset, wherein each of a plurality of position vectors in the position vector dataset is associated with at least one signal parameter obtained using a known position of the object. For instance, RFID reader 320 can be used to obtain at least one signal parameter from RF ID tag 304a and/or 304b. In some aspects, RFID reader 320 can obtain a position vector dataset that includes position vectors 308, 310, 312, 314, 316, and 318. In some examples, each position vectors can be associated with a signal parameter (e.g., RSSI, phase, etc.) obtained using a known position of surgical instrument 302 (e.g., position 306a, 306b, and/or 306c). In some cases, the known position of the object can be based on a robotic arm position. For example, robot 204 may position surgical instrument 302 in one or more known positions and/or one or more known orientations.

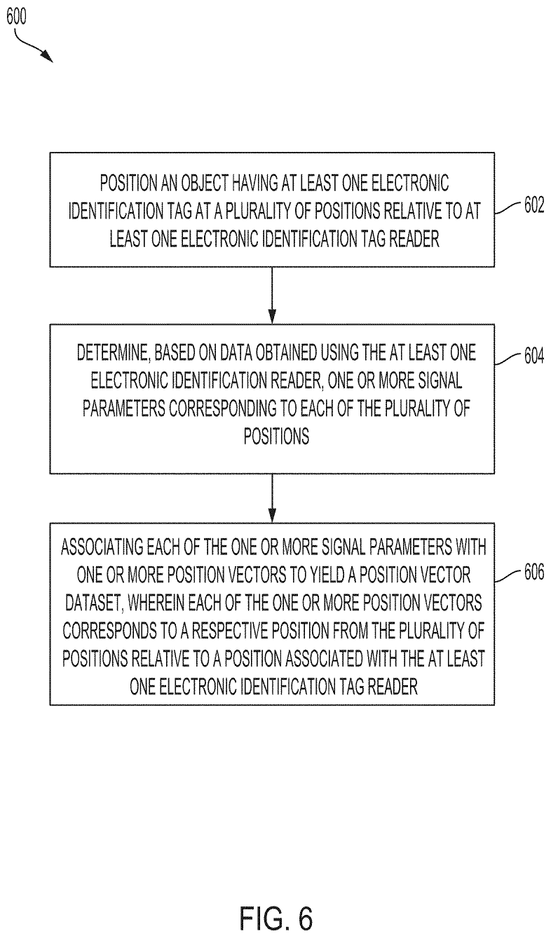

FIG. 6 illustrates an example method 600 for training a machine learning model to locate objects based on RFID data. At block 602, the method 600 includes positioning an object having at least one electronic identification tag at a plurality of positions relative to at least one electronic identification reader. For instance, surgical instrument 302 can have RFID tag 304a and 304b, and surgical instrument 302 can be positioned at position 306a, 306b, and/or 306c relative to RFID reader 320 at position 322.

At block 604, the method 600 includes determining, based on data obtained using the at least one electronic identification reader, one or more signal parameters corresponding to each of the plurality of positions. For instance, RFID reader 320 can determine one or more signal parameters corresponding to surgical instrument at one or more of positions 306a, 306b, and/or 306c. In some aspects, the one or more parameters can include at least one of a phase, a frequency, a received signal strength indicator (RSSI), a time of flight (ToF), an Electronic Product Code (EPC), and an instrument geometry identifier.

At block 606, the method 600 includes associating each of the one or more signal parameters with one or more position vectors to yield a position vector dataset, wherein each of the one or more position vectors corresponds to a respective position from the plurality of positions relative to a position associated with the at least one electronic identification tag reader. For instance, one or more RFID parameters obtained using RFID reader 320 can be associated with one or more of position vectors 308, 310, 312, 314, 316, and 318. In some aspects, each position vector can correspond to a respective position for surgical instrument 302 relative to a position for RFID reader 320 (e.g., position vector 308 corresponds to position 306a for RFID tag 304a relative to RFID reader 320 at position 322.

In some embodiments, the method 600 may include training the machine learning algorithm using the position vector dataset. In some cases, the machine learning algorithm can correspond to a Gaussian Process Regression algorithm. In some examples, the positioning of the object can be performed using a robotic arm. For instance, robot 204 can position surgical instrument 210. In some aspects, the object can include at least one of a medical device and a surgical instrument (e.g., surgical instrument 210).

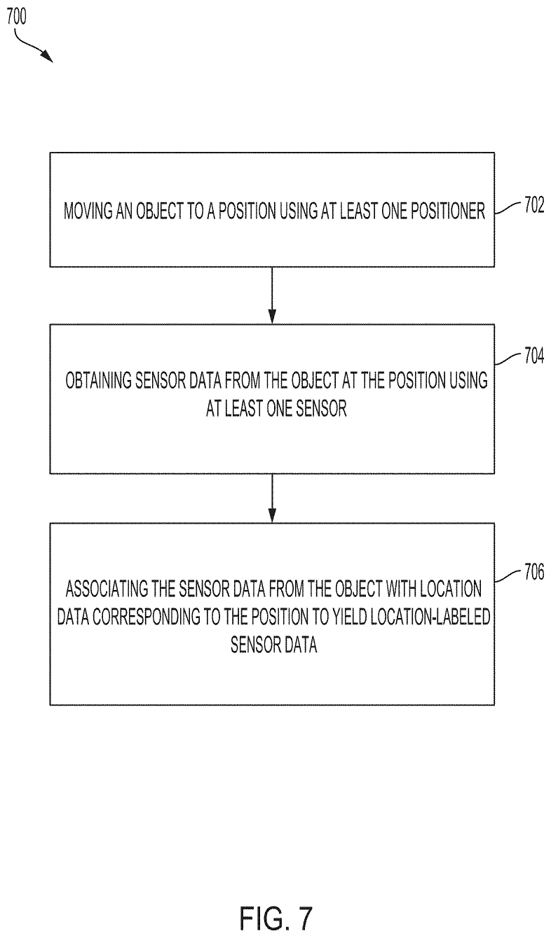

FIG. 7 illustrates an example method 700 for locating objects. At block 702, the method 700 includes moving an object to a position using at least one positioner. In some aspects, the position of the object can be based on a robotic position. For instance, robot 204 can position surgical instrument 302 at position 306a. In some cases, the at least one positioner may include a string localizer (e.g., including one or more stepper motors and spools of string that may be tied to an object).

At block 704, the method 700 includes obtaining sensor data from the object at the position using at least one sensor. In some cases, the sensor data can include at least one of a phase, a frequency, a received signal strength indicator (RSSI), a time of flight (ToF), an Electronic Product Code (EPC), a time-to-read, an image, and an instrument geometry identifier. In some aspects, the at least one sensor can include at least one of a radio frequency identification (RFID) reader, a camera, and a stereo camera.

At block 706, the method 700 includes associating the sensor data from the object with location data corresponding to the position to yield location-labeled sensor data. In some embodiments, the object can include at least one of a medical device and a surgical instrument. For example, the object can include surgical instrument 210. In some cases, the object can be associated with an electronic identification tag. For instance, surgical instrument 210 is associated with RFID tag 212a and RFID tag 212b.

In some aspects, a machine learning algorithm can be trained using the location-labeled sensor data to yield a trained machine learning algorithm. For example, the location-labeled sensor data can be stored in a database and used to train and test a machine learning algorithm. In some configurations, the trained machine learning algorithm can be used to process new sensor data collected in a new environment, wherein the new environment is different that a first environment associated with the system. For instance, system 200 can be used to train a machine learning algorithm to detect and/or locate objects. In some cases, the new environment can correspond to an operating room and the new sensor data can correspond to data obtained from at least one surgical instrument. For example, the machine learning algorithm can be used in an environment such as OR 101 to process sensor data associated with one or more objects such as surgical instrument 145.

In some examples, the process 700 can include rotating the object about at least one axis at the position. For example, a robotic arm (e.g., robot 204) can be used to rotate surgical instrument 210 about an axis while surgical instrument 210 is located at a same position. In some cases, rotation of an object can be used to change the orientation of the object. In some instances, sensor data (e.g., RFID parameters) can be collected during rotation of an object and/or after the object is rotated.

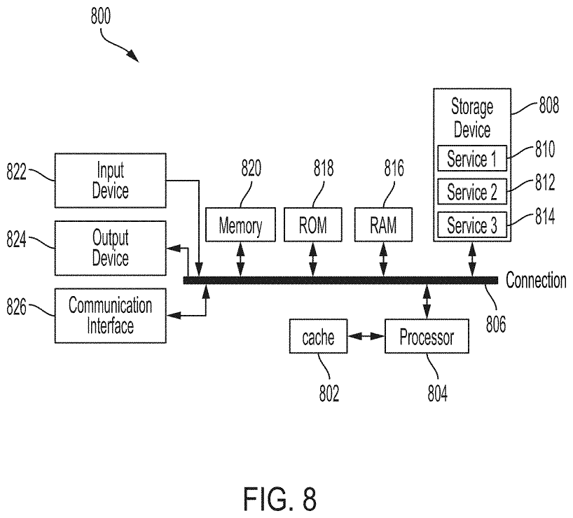

FIG. 8 illustrates an example computing system 800 for implementing certain aspects of the present technology. In this example, the components of the system 800 are in electrical communication with each other using a connection 806, such as a bus. The system 800 includes a processing unit (CPU or processor) 804 and a connection 806 that couples various system components including a memory 820, such as read only memory (ROM) 818 and random access memory (RAM) 816, to the processor 804.

The system 800 can include a cache of high-speed memory connected directly with, in close proximity to, or integrated as part of the processor 804. The system 800 can copy data from the memory 820 and/or the storage device 808 to cache 802 for quick access by the processor 804. In this way, the cache can provide a performance boost that avoids processor 804 delays while waiting for data. These and other modules can control or be configured to control the processor 804 to perform various actions. Other memory 820 may be available for use as well. The memory 820 can include multiple different types of memory with different performance characteristics. The processor 804 can include any general purpose processor and a hardware or software service, such as service 1 810, service 2 812, and service 3 814 stored in storage device 808, configured to control the processor 804 as well as a special-purpose processor where software instructions are incorporated into the actual processor design. The processor 804 may be a completely self-contained computing system, containing multiple cores or processors, a bus, memory controller, cache, etc. A multi-core processor may be symmetric or asymmetric.

To enable user interaction with the computing system 800, an input device 822 can represent any number of input mechanisms, such as a microphone for speech, a touch-sensitive screen for gesture or graphical input, keyboard, mouse, motion input, speech and so forth. An output device 824 can also be one or more of a number of output mechanisms known to those of skill in the art. In some instances, multimodal systems can enable a user to provide multiple types of input to communicate with the computing system 800. The communications interface 826 can generally govern and manage the user input and system output. There is no restriction on operating on any particular hardware arrangement and therefore the basic features here may easily be substituted for improved hardware or firmware arrangements as they are developed.

Storage device 808 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 816, read only memory (ROM) 818, and hybrids thereof.

The storage device 808 can include services 810, 812, 814 for controlling the processor 804. Other hardware or software modules are contemplated. The storage device 808 can be connected to the connection 806. In one aspect, a hardware module that performs a particular function can include the software component stored in a computer-readable medium in connection with the necessary hardware components, such as the processor 804, connection 806, output device 824, and so forth, to carry out the function.

It is to be understood that the systems described herein can be implemented in hardware, software, firmware, or combinations of hardware, software and/or firmware. In some examples, image processing may be implemented using a non-transitory computer readable medium storing computer executable instructions that when executed by one or more processors of a computer cause the computer to perform operations. Computer readable media suitable for implementing the control systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements an image processing system described in this specification may be located on a single device or computing platform or may be distributed across multiple devices or computing platforms.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A system comprising:
at least one memory;
at least one sensor;
at least one positioner; and
at least one processor coupled to the at least one memory, the at least one sensor, and the at least one positioner, wherein the at least one processor is configured to:
move an object to a position using the at least one positioner;
obtain sensor data from the object at the position using the at least one sensor; and
associate the sensor data from the object with location data corresponding to the position to yield location-labeled sensor data.

2. The system of claim 1, wherein a machine learning algorithm is trained using the location-labeled sensor data to yield a trained machine learning algorithm.

3. The system of claim 2, wherein the trained machine learning algorithm is used to process new sensor data collected in a new environment, wherein the new environment is different than a first environment associated with the system.

4. The system of claim 3, wherein the new environment corresponds to an operating room, and wherein the new sensor data corresponds to data obtained from at least one surgical instrument.

5. The system of claim 1, wherein the position of the object is based on a robotic position.

6. The system of claim 1, wherein the at least one sensor includes at least one of a radio frequency identification (RFID) reader, a camera, and a stereo camera.

7. The system of claim 1, wherein the sensor data includes at least one of a phase, a frequency, a received signal strength indicator (RSSI), a time of flight (ToF), an Electronic Product Code (EPC), a time-to-read, an image, and an instrument geometry identifier.

8. The system of claim 1, wherein the object includes at least one of a medical device and a surgical instrument, and wherein the object is associated with an electronic identification tag.

9. The system of claim 1, wherein the at least one processor is further configured to:
rotate the object about at least one axis at the position.

10. A system comprising:
at least one memory;
at least one transceiver; and
at least one processor coupled to the at least one memory and the at least one transceiver, the at least one processor configured to:
receive, via the at least one transceiver, at least one radio frequency (RF) signal from an electronic identification tag associated with an object;
determine one or more parameters associated with the at least one RF signal; and
process the one or more parameters with a machine learning algorithm to determine a position of the object.

11. The system of claim 10, wherein the machine learning algorithm is trained using a position vector dataset, wherein each of a plurality of position vectors in the position vector dataset is associated with at least one signal parameter obtained using a known position of the object.

12. The system of claim 11, wherein the known position of the object is based on a robotic arm position.

13. The system of claim 10, wherein the one or more parameters include at least one of a phase, a frequency, a received signal strength indicator (RSSI), a time of flight (ToF), an Electronic Product Code (EPC), and an instrument geometry identifier.

14. The system of claim 10, wherein the object includes at least one of a medical device and a surgical instrument, and wherein the object is within an operating room environment.

15. The system of claim 10, wherein the electronic identification tag is a radio frequency identification (RFID) tag.

16. A method of locating objects, comprising:
receiving at least one radio frequency (RF) signal from an electronic identification tag associated with an object;
determining one or more parameters associated with the at least one RF signal; and
processing the one or more parameters with a machine learning algorithm to determine a position of the object.

17. The method of claim 16, wherein the machine learning algorithm is trained using a position vector dataset, wherein each of a plurality of position vectors in the position vector dataset is associated with at least one signal parameter obtained using a known position of the object.

18. The method of claim 17, wherein the known position of the object is based on a robotic arm position.

19. The method of claim 16, wherein the one or more parameters include at least one of a phase, a frequency, a received signal strength indicator (RSSI), a time of flight (ToF), an Electronic Product Code (EPC), and an instrument geometry identifier.

20. The method of claim 16, wherein the object includes at least one of a medical device and a surgical instrument, and wherein the object is within an operating room environment.

21. A method of training a machine learning algorithm, comprising:
positioning an object having at least one electronic identification tag at a plurality of positions relative to at least one electronic identification tag reader;
determining, based on data obtained using the at least one electronic identification tag reader, one or more signal parameters corresponding to each of the plurality of positions; and
associating each of the one or more signal parameters with one or more position vectors to yield a position vector dataset, wherein each of the one or more position vectors corresponds to a respective position from the plurality of positions relative to a position associated with the at least one electronic identification tag reader.

22. The method of claim 21, further comprising:
training the machine learning algorithm using the position vector dataset.

23. The method of claim 21, wherein the positioning is performed using a robotic arm.

24. The method of claim 21, wherein the one or more signal parameters include at least one of a phase, a frequency, a received signal strength indicator (RSSI), a time of flight (ToF), an Electronic Product Code (EPC), and an instrument geometry identifier.

25. The method of claim 21, wherein the object includes at least one of a medical device and a surgical instrument.
